(19) United States
(12) Patent Application Publication
(10) Pub. No.: US 2021/0241040 A1
(43) Pub. Date: Aug. 5, 2021

(54) Systems and Methods for Ground Truth Dataset Curation

(71) Applicant: Origin Labs, Inc., San Francisco, CA (US)

(72) Inventors: Darick M. Tong, San Francisco, CA (US); Evan Szu, Zephyr Cove, NV (US); Clifford Szu, Zephyr Cove, NV (US); Nishant Borude, San Francisco, CA (US); Nivedita Suresh, San Francisco, CA (US)

(21) Appl. No.: 17/168,704

(22) Filed: Feb. 5, 2021

Related U.S. Application Data
(60) Provisional application No. 62/970,314, filed on Feb. 5, 2020.

(51) Int. Cl. G06K 9/62; G06N 20/00; G16H 30/40

(52) U.S. Cl. CPC G06K 9/6257; G16H 30/40; G06N 20/00; G06K 9/627

(57) ABSTRACT

Systems and methods of the present disclosure enable ground truth dataset creation using processing devices configured to receive at least one user profile of at least one user and user-generated labels for each sample of at least one sample, where the user-generated labels are associated with the at least one user profile. A most likely label for each sample is produced using a weighting of each user-generated label according to user attributes in each user profile associated with each user-generated label. Each most likely label is recorded for each sample in a ground-truth dataset associated with each sample to identify a ground truth of the at least one sample, and each most likely label and each sample is provided to a machine learning model to train the machine learning model according to a difference between a predicted label for each sample and each most likely label.

### RELATED APPLICATIONS

This application claims priority to and the benefit of U.S. Provisional Patent Application No. 62/970,314, filed Feb. 5, 2020, which is incorporated by reference herein in its entirety.

### COPYRIGHT NOTICE

A portion of the disclosure of this patent document contains material that is subject to copyright protection. The copyright owner has no objection to the facsimile reproduction by anyone of the patent document or the patent disclosure, as it appears in the Patent and Trademark Office patent files or records, but otherwise reserves all copyright rights whatsoever. The following notice applies to the software and data as described below and in drawings that form a part of this document: Copyright, Origin Labs, Inc., All Rights Reserved.

### FIELD

The present disclosure generally relates to improved computer-based systems configured for ground truth dataset curation, including determining ground truth data in sets for training machine learning models.

### BACKGROUND

Many fields in medicine and pharmacology depend on obtaining ground truth data, including in the space of training machine learning systems to automatically recognize this type of data. Many machine learning models, especially supervised, semi-supervised and weakly supervised machine learning models, are trained using labeled data that are part of a training set. The accuracy of a model is often limited by the quantity and quality of training data used to train the model. The more accurate data a training set includes, oftentimes the more accurate a model can be. Moreover, the accuracy of the model is limited by the quality of the training data used to train the model. If the training data used to train the model is inaccurate, the resulting model will also likely be inaccurate.

For many applications, such as to train whether an image depicts a specific species of animal, a vast amount of accurate training data is available. However, for other applications, such as for determining whether an image indicated the presence of a tumor, the available training data is very limited. Moreover, since the assessment of multiple doctors of the same image may be different, the quality of the training data is oftentimes low.

### BRIEF DESCRIPTION OF THE DRAWINGS

Various embodiments of the present disclosure can be further explained with reference to the attached drawings, wherein like structures are referred to by like numerals throughout the several views. The drawings shown are not necessarily to scale, with emphasis instead generally being placed upon illustrating the principles of the present disclosure. Therefore, specific structural and functional details disclosed herein are not to be interpreted as limiting, but merely as a representative basis for teaching one skilled in the art to variously employ one or more illustrative embodiments.

FIG. 1 is a block diagram of an exemplary computer-based system for curating a ground truth dataset from user-generated labels in sample data in accordance with one or more embodiments of the present disclosure.

FIG. 2, FIG. 3, FIG. 4, FIG. 5, FIG. 6, FIG. 7 and FIG. 8 show one or more schematic flow diagrams, certain computer-based architectures, and/or screenshots of various specialized graphical user interfaces which are illustrative of some exemplary aspects of at least some embodiments of the present disclosure.

### DETAILED DESCRIPTION

Various detailed embodiments of the present disclosure, taken in conjunction with the accompanying figures, are disclosed herein; however, it is to be understood that the disclosed embodiments are merely illustrative. In addition, each of the examples given in connection with the various embodiments of the present disclosure is intended to be illustrative, and not restrictive.

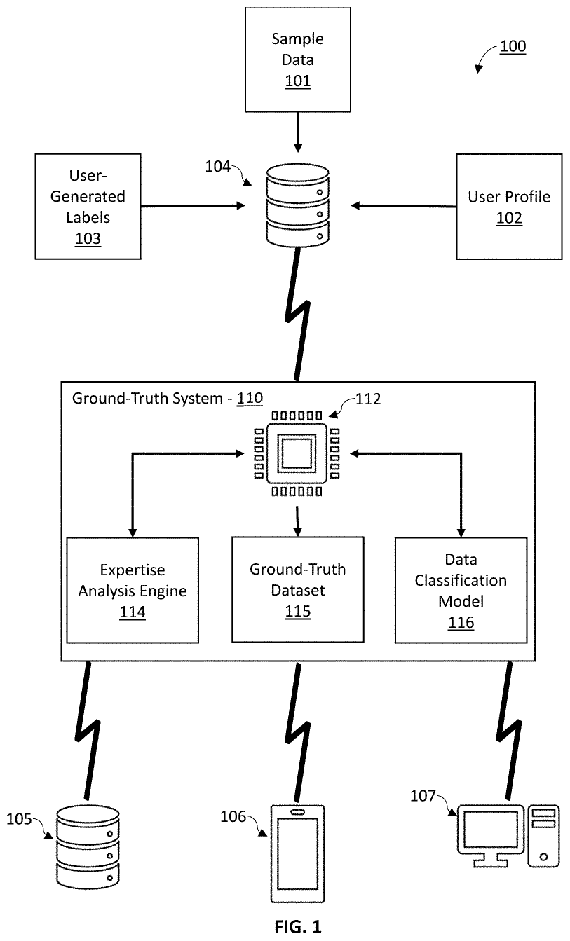

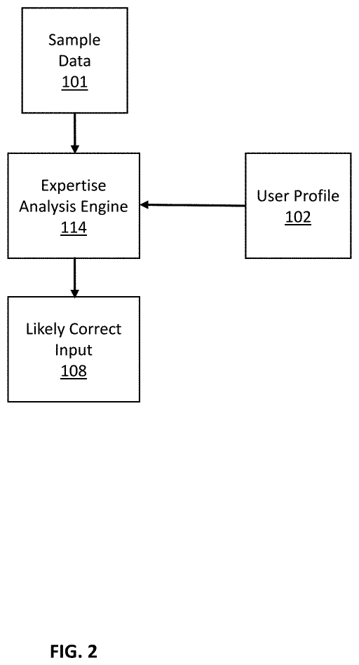

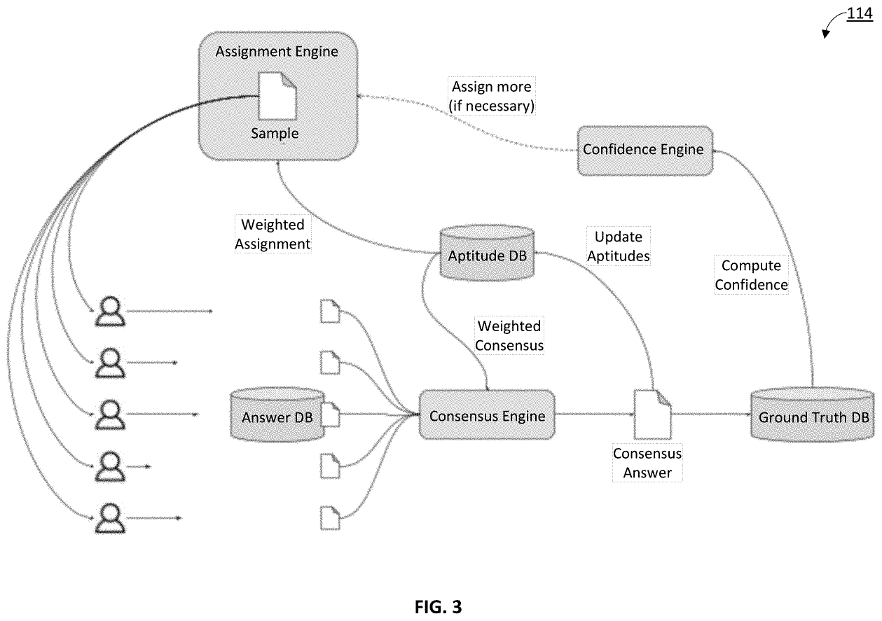

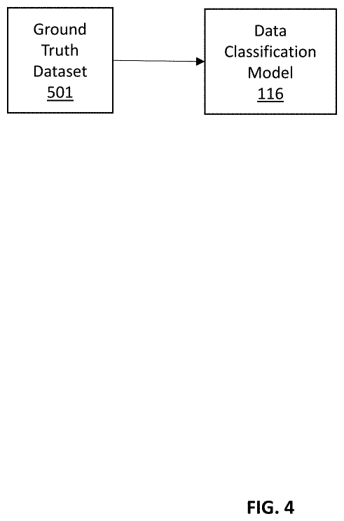

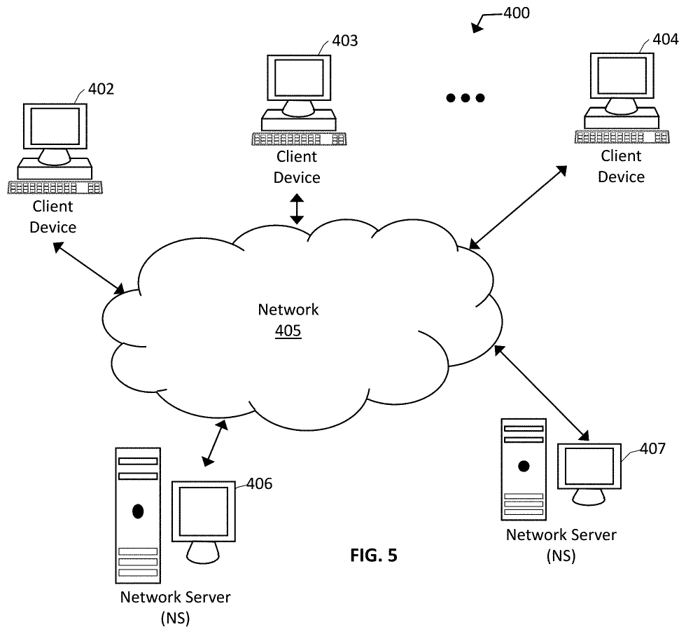

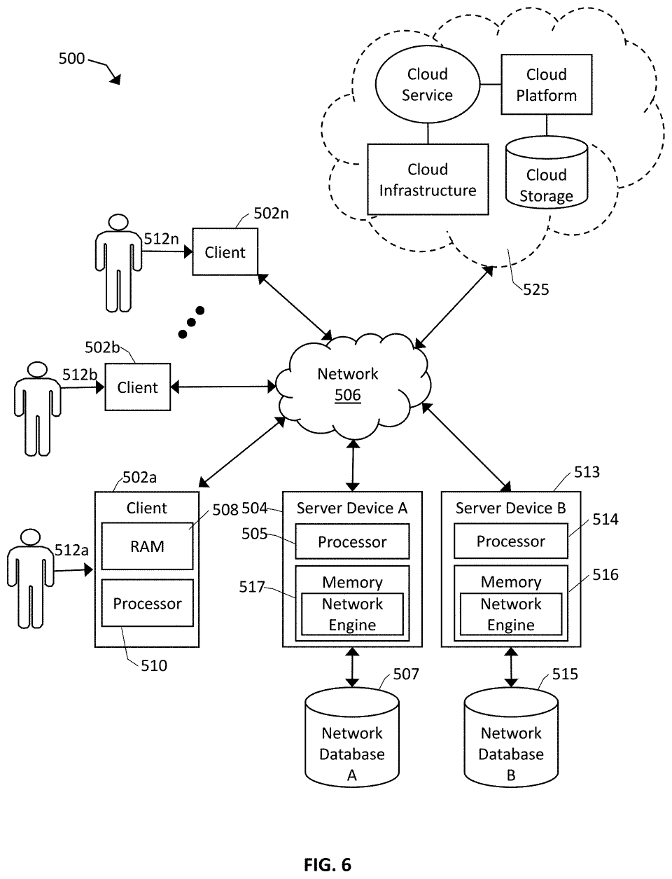

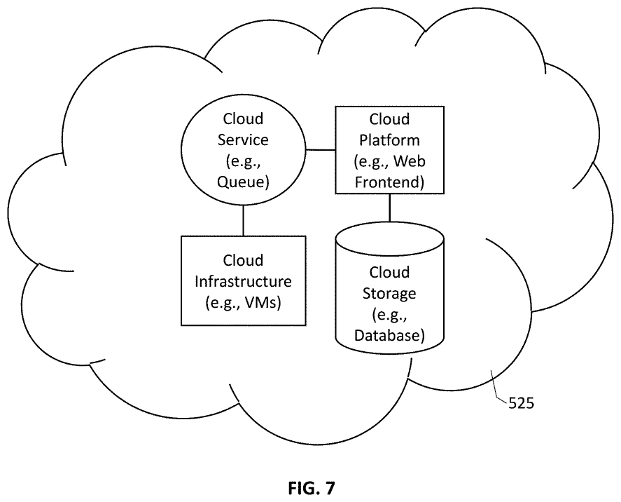

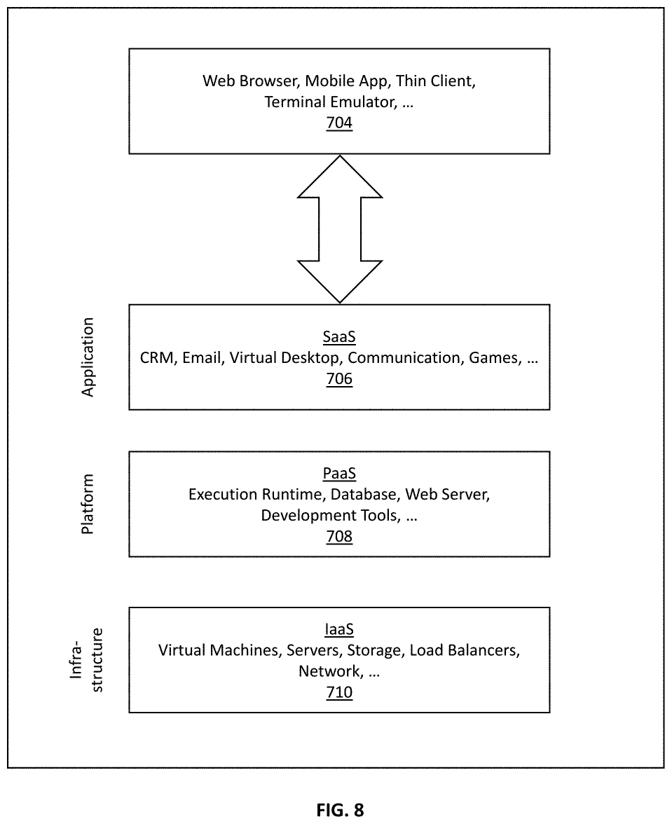

FIG. 1, FIG. 2, FIG. 3, FIG. 4, FIG. 5, FIG. 6, FIG. 7 and FIG. 8 illustrate systems and methods of identifying and generating reliable datasets for training machine learning models. The following embodiments provide technical solutions and technical improvements that overcome technical problems, drawbacks and/or deficiencies in the technical fields involving data classification tasks, such as the classification of data obtained from imagery, audio samples, language transcription samples, among other data for classification, such as, e.g., biological samples, including medical imagery, chemical data, multi-omics data, numerical or sensor data, among others. As explained in more detail below, technical solutions and technical improvements herein include aspects of improved data classification and improved machine learning training for more accurate and efficient data classification through automatic mitigations of inaccuracies in human annotations. Based on such technical features, further technical benefits become available to users and operators of these systems and methods. Moreover, various practical applications of the disclosed technology are also described, which provide further practical benefits to users and operators that are also new and useful improvements in the art.

FIG. 1 is a block diagram of an exemplary computer-based system for curating a ground truth dataset from user-generated labels in sample data in accordance with one or more embodiments of the present disclosure.

In some embodiments, an illustrative data classification system 100 provides a way of gathering labeled training data for the classification of data from audio, visual, image, text and/or data samples, e.g., audio transcriptions, audio data, one or more images, scientific testing and analysis data, text documents and text strings, biological samples, among other data samples or any combination thereof. In some embodiments, the availability of trained individuals that are capable of providing an assessment of the data sample is low, and the concordance between the assessment of multiple individuals vary. The data classification system 100 collects inputs from users to determine ground truth for data samples of any particular type, such as, e.g., biological sample data.

In some embodiments, the sample data 101 and user-generated labels 103 may be provided, e.g., via a network from a user device, such as, e.g., user devices 105-107, among others. In some embodiments, the sample data 101 and user-generated labels 103 may be provided via a cloud platform, such as, e.g., downloaded or streamed from a cloud storage network, accessed via the cloud platform, or other cloud architecture. In some embodiments, the sample data 101 and user-generated labels 103 may be physically transferred, e.g., on a hard drive provided to and attached to the ground-truth system 110 to transfer copies of the samples and user input.

In some embodiments, the user-generated labels 103 may be recorded, e.g., using a suitable input device, such as, e.g., a keyboard, a pointing device including a mouse or trackpad, via a touchscreen input, gaze tracking, or other suitable input at, e.g., a user device of the user devices 105-107. In some embodiments, the user inputs may be encrypted and stored, e.g., in a cloud storage or on-premises, e.g., in a hard drive, database, solid-state drive, or other storage device or system, such as the database 105 of the user devices 105-107.

In some embodiments, the ground-truth system 110 may select users based on user profile 102 to match one or more criteria in a data sample. The criteria may depend on the type of data sample. For example, the criteria may include, e.g., languages known, degree of fluency, number of translations performed, and the like for ground truth translation data sets. For another example, for classification for sentiment analysis of text (e.g., in social media posts, blogs, articles, etc. of companies, securities, commodities, etc.), the criteria may include, e.g., degrees held, sentiment analysis history, etc. In another example, for audio transcription (e.g., to train voice recognition models), the criteria may include, e.g., degrees obtained, certifications, employment history in transcription and dictation related fields, etc.

In some embodiments, the ground-truth system 110 may select users based on user profile 102 to match one or more criteria in a patient for biological sample classification, e.g., credit score, degrees attained, years of work experience, etc. In some embodiments, data, such as user profile 102 information and patient information, can be identified or de-identified automatically in the system using machine learning visual recognition of proper names and other personally identifiable data.

As such, in some embodiments, a ground-truth system 110 accesses sample data 101 and associated user-generated labels 103 classifying the sample data 101 from data storage 104.

In some embodiments, the data storage 104 may include, e.g., a suitable memory or storage solutions for providing electronic data to the ground-truth system 110. For example, the data storage 104 may include, e.g., a centralized or distributed database, cloud storage platform, decentralized system, server or server system, among other storage systems, or the data storage 104 may include, e.g., a hard drive, solid-state drive, flash drive, or other suitable storage device, or the data storage 104 may include, e.g., a random access memory, cache, buffer, or other suitable memory device, or any other data storage solution and combinations thereof.

In some embodiments, the ground-truth system 110 receives the user-generated labels 103 and the sample data 101 and identifies the most likely correct user-generated labels 103. In some embodiments, the sample data 101 may include, e.g., biological sample data, audio data for transcription, text data for translation, text data for sentiment analysis, imagery for classification and object detection, and any other suitable sample data 101. For example, the sample data 101 may be image data, such as medical imagery or microscopy images, or subsets of images. In some embodiments, the sample data 101 may be chemical data, multi-omics data, or numerical data such as sensor data. Other types of sample data 101 are also contemplated.

In some embodiments, to facilitate identifying the most likely correct user-generated labels 103, e.g., ground-truth labels, for each data item in the sample data 101, the ground-truth system 110 may utilize user profiles 102 of the users providing the user-generated labels 103. Because the user-generated labels 103 may vary across different users, ground-truth labels can be difficult to identify. However, users having more expertise in a given field may be more likely to provide more accurate user-generated labels 103.

In some embodiments, the practice field or field of expertise needed for a sample in the sample data 101 may be identified, e.g., via visual pattern recognition. For example, a machine learning model, such as deep learning (e.g., deep neural network or other neural network architecture) may be employed for pattern recognition in the sample data 101 to identify types of samples and associated fields of practice. In some embodiments, the practice field may be identified through analysis of file types and/or metadata associated with the sample data 101. In some embodiments, a human user may categorize the samples of the sample data 101. In some embodiments, the practice fields may be recognized using the files, metadata, human categorization or other suitable method or any combination thereof.

In some embodiments, the machine learning model may include, e.g., a computer vision model trained to classify medical imagery according to, e.g., a type of tissue. In some embodiments, the machine learning model may include natural language recognition or other forms of semantic segmentation of text to identify topics and concepts, among other automated recognition tasks. In some embodiments, the automated recognition can be used to define a filtering element to match users with expertise according to the filter. Any other automated incarnation of patient selection may be employed.

Thus, the ground-truth system 110 may assess the expertise of each user based on user profiles 102, e.g., received via the data storage 104, using an expertise analysis engine 114. Accordingly, in some embodiments, the system may first determine the skill levels of users and use a single user per data point for determining ground truth based on a user profile 102 indicating a greatest degree of expertise, and thus most likely accurate user-generated label 103 for a given sample data 101 item. For example, the expertise analysis engine 114 implements the process discussed with respect to FIG. 2 and FIG. 3 to assess the expertise of a user.

In some embodiments, the expertise analysis engine 114 may utilize the user profile 102 to determine the most likely correct user-generated label 103 based on one or more of multiple factors recorded in the user profile 102, including, but not limited to: user experience, number of user contributions, educational or work background, psychometric test scores, system account analysis and analysis of third party accounts, qualitative or quantitative skill level evaluations, publishing history and/or citation history, prior user inputs, ratings from other users, ratings from non-users, industry peers and/or domain experts, consensus with other users, behavioral analysis, user-provided decision rationale inputs, comparison against existing data, and financial scores and scores from third party rating services, among other factors and combinations thereof. In some embodiments, this information is stored in an encrypted database either locally or in the cloud. In some embodiments, the ground-truth system 110 learns over time which parameters are the most important for obtaining the optimum quality for any practice area and data type and ranks users with incomplete or missing data using alternative fields in the list (e.g., with a potential penalty for missing user-generated data).

In some embodiments, based on the user having the greatest expertise for a given sample data 101, the user-generated label associated with that user of the user-generated labels 103 may be selected as the ground-truth label for the given sample data 101. In some embodiments, one or more user-generated labels 103 may be used as ground-truth for a given sample of the sample data 101. In some embodiments, e.g., in the case of final target data existing in floating point format, user scores may be statistically aggregated, such as, e.g., averaged or averaged in a weighted fashion, a median or weighted median or the like, where the weighting is based on the skill level of the users providing the scores. For example, in the case of integral data, a final single value or a set of values with confidence intervals for each value may be generated if multiple users are contributing information. This data classification process may be performed in an asynchronous way, where each of the users is able to provide their assessment of the data or a subset of the data independently of the other users.

In some embodiments, there may be a predetermined time period in which users may submit user-generated labels 103 to create a final score indicative of a label for a sample in the sample data 101. However, in some embodiments, the final score may be re-evaluated periodically if additional users are assigned to generate labels 103. In some embodiments, once a confidence score of the final score attains a threshold degree of confidence, e.g., about 0.7, about 0.8, about 0.9, about 0.95 or other degree of confidence, the score and label may be finalized as ground-truth. Moreover, the classification process may be blind (e.g., each user is not informed of how other users have voted for a given data point of the sample data 101), where none of the users know the assessment of other users before providing an answer.

In some embodiments, each ground-truth label identified by the expertise analysis engine 114 may be assigned to a corresponding sample data 101 item to create a ground-truth data record for the sample data 101 item. Thus, the user-generated labels 103 may be filtered by the expertise analysis engine 114 to identify the one label of the user-generated labels 103 for each sample data 101 item that is most likely to be correct, and add the label to a ground-truth dataset 115.

In some embodiments, the ground-truth dataset 115 may be used to train a data classification model 116 that classifies sample data 101 automatically. In some embodiments, the data classification model 116 may be configured to utilize one or more exemplary AI or machine learning techniques chosen from, but not limited to, decision trees, boosting, support-vector machines, neural networks, nearest neighbor algorithms, Naive Bayes, bagging, random forests, and the like. In some embodiments and, optionally, in combination of any embodiment described above or below, an exemplary neural network technique may be one of, without limitation, feedforward neural network, radial basis function network, recurrent neural network, convolutional network (e.g., U-net) or other suitable network. In some embodiments and, optionally, in combination of any embodiment described above or below, an exemplary implementation of neural network may be executed as follows:

i) define neural network architecture/model,

ii) transfer the input data to the exemplary neural network model,

iii) train the exemplary model incrementally,

iv) determine the accuracy for a specific number of timesteps,

v) apply the exemplary trained model to process the newly-received input data,

vi) optionally and in parallel, continue to train the exemplary trained model with a predetermined periodicity.

In some embodiments and, optionally, in combination of any embodiment described above or below, the exemplary trained neural network model may specify a neural network by at least a neural network topology, a series of activation functions, and connection weights. For example, the topology of a neural network may include a configuration of nodes of the neural network and connections between such nodes. In some embodiments and, optionally, in combination of any embodiment described above or below, the exemplary trained neural network model may also be specified to include other parameters, including but not limited to, bias values, functions and aggregation functions. For example, an activation function of a node may be a step function, sine function, continuous or piecewise linear function, sigmoid function, hyperbolic tangent function, or other type of mathematical function that represents a threshold at which the node is activated. In some embodiments and, optionally, in combination of any embodiment described above or below, the exemplary aggregation function may be a mathematical function that combines (e.g., sum, product, etc.) input signals to the node. In some embodiments and, optionally, in combination of any embodiment described above or below, an output of the exemplary aggregation function may be used as input to the exemplary activation function. In some embodiments and, optionally, in combination of any embodiment described above or below, the bias may be a constant value or function that may be used by the aggregation function and/or the activation function to make the node more or less likely to be activated.

Thus, in some embodiments, the ground-truth dataset 115 may be used to adjust the aggregation or activation function of the data classification model 116 to better predict labels for sample data 101 without human intervention based on an error of the prediction from the ground-truth dataset 115. Because the expertise analysis engine 114 maximizes the accuracy of the provided user-generated labels 103, the ground-truth dataset 115 is most likely to be accurate, despite low amounts of expert users. As a result, the data classification model 116 is made more accurate due to accurate training data, thus improving the efficiency of training the data classification model 116 to achieve accuracy, while also achieving greater accuracy.

In some embodiments, the expertise analysis engine 114 and the data classification model 116 may include at least one software component and/or a combination of at least one software component and at least one hardware component which are designed/programmed/configured to manage/control other software and/or hardware components (such as the libraries, software development kits (SDKs), objects, etc.).

In some embodiments, the expertise analysis engine 114 and the data classification model 116 may be implemented and/or executed by a processing device 112. In some embodiments, the processing device may include any type of data processing capacity, such a hardware logic circuit, for example, an application specific integrated circuit (ASIC) and a programmable logic, or such as a computing device, for example, a microcomputer or microcontroller that include a programmable microprocessor. In some embodiments, the processing device may include data-processing capacity provided by the microprocessor. In some embodiments, the microprocessor may include memory, processing, interface resources, controllers, and counters. In some embodiments, the microprocessor may also include one or more programs stored in memory.

In some embodiments, the ground-truth system 110 may output the ground-truth dataset 115, data classifications from the data classification model 116, among other data. In some embodiments, the ground-truth system 110 may be in communication with a ground-truth dataset 115 and/or the data classifications to a database 105 for persistent storage. In some embodiments, the database 105 can include, e.g., centralized or distributed database, one or more servers, a cloud storage platform, or other data storage system and combinations thereof.

Additionally, the ground-truth system 110 may be in communication with a user mobile device 106 or user computing device 107. A user may interact with the ground-truth system 110 via the user mobile device 106 or the user computing device 107 to, e.g., view data classifications, view and modify the ground-truth dataset 115, input user-generated labels 103 for sample data 101 using an associated user profile 102, among other functions and interactions.

FIGS. 2 and 3 are block diagrams of the expertise analysis engine 114 and methods for expertise analysis in accordance with one or more embodiments of the present disclosure.

In some embodiments, the expertise analysis engine 114 weights labels for sample data 101 associated with each user profile 102 based on user attributes of the user in the user profile 102. For example, the expertise analysis engine 114 may use machine learning and test data sets to gradually determine the most relevant data sources for each type of classification task. For example, for a pathology task, years of experience may be more relevant, but for a simpler task, the amount of time the user has owned an account on our system may be more relevant. The deep learning system gradually adjusts weights of each field until optimal accuracy is reached when given a sufficient amount of data. In the situation where no data exists, transfer learning may occur where data is imported from parallel or similar tasks (e.g. cytology weights can be imported into cell biology expertise analysis engine weights) or simply a default set of weights (e.g. all existing weights across all fields aggregated, e.g., determine an average, a weighted average, a median, a weighted median, a sum, a weighted sum, etc.). Based on the weighting of each user input, e.g., each label or score provided by each user, a likely correct input 108 may be determined that, e.g., forms a weighted sum, weighted average, or other suitable weighted aggregation of user inputs, to use as ground-truth for training the data classification model 116.

In some embodiments, weights according to user skill in the type of classification of the sample data 101 includes quantifying “distance from consensus” and using a distance measure to derive and quantify the skill of a user. Users that tend to be closer to consensus are considered to have a higher aptitude/skill and are thus given more weight when evaluating consensus rounds in future samples.

In some embodiments, the expertise analysis engine 114 may include an assignment engine, a confidence engine and a consensus engine to identify the consensus, the distance from the consensus and the resulting weight of each user. In some embodiments, the assignment engine, confidence engine and consensus engine cooperate to represent the solution space in a fashion that facilitates computing a mathematical average or other statistical aggregation (e.g., sum, median, product, etc.). For linear tasks (e.g. sentiment, scoring, “on a scale of 0 to 100”, etc.) this is straightforward. For discrete tasks such as N-class classification (e.g. Tumor, Immune, Stromal), answers can be represented as unit vectors in an N-dimensional space.

In some embodiments, the assignment engine may match the sample data 101 of a sample to profiles of users, as described above, to assign a labelling task to one or more matching users. In some embodiments, the consensus engine determines a consensus answer among the matched user answers. In some embodiments, the consensus answer is a weighted average of all user inputs, whereby the “weights” are (1) derived from performance in previous consensus rounds, and (2) incorporate external measurements of user skill such as, e.g., “degrees held” and “years of experience” among others including those described above. In some embodiments, the external measurements may be omitted, but inclusion can facilitate bootstrapping the algorithm and/or safeguarding against erroneous user labels (e.g., due to abuse of the system, gaming the ground truth data, etc.).

In some embodiments, the consensus answer may be stored in the ground truth database, e.g., to form the ground-truth dataset 115. In some embodiments, for each consensus answer, the users' accuracies are determined according to a distance, e.g., the Euclidean distance, from each answer to the consensus answer. For example, (ignoring weights for simplicity) if 9 users answer “10” and 1 user answers “0”, the consensus answer is “9” and the last user's accuracy is much lower than those of the first 9.

In some embodiments, each user's accuracies may then be averaged across a history of samples, producing an Aptitude. In some embodiments, each user's Aptitude may be stored in the Aptitude database. The aptitudes may then be used as the weight for future consensus rounds. A user that is usually closer to consensus will have higher weight than one who is usually further. When this computation is performed iteratively over many samples, Aptitudes converge.

In some embodiments, the confidence engine may use the user answers including user labels for the sample data as well as the consensus answer to determine a confidence of the consensus answer. In some embodiments, because a consensus answer is computed from the (weighted) mean of a population of answers, the confidence of a consensus answer is derived from a computation analogous to the standard error of the mean. For example, using the current population of answers as the sampling distribution, simulations may be run by the confidence engine to compute hypothetical means of populations drawn from a similar distribution. For example, with (1) each sample removed and (2) each sample doubled. This produces a distribution of means (i.e. consensus answers) for which the standard deviation reflects the confidence in the original consensus answer.

In some embodiments, as the sample size increases, each sample has less of an effect on the mean when it is removed or doubled. Therefore, it follows that the standard error decreases (and confidence increases) as the sample size increases.

In some embodiments, for continuous solution spaces (e.g. 0 to 100), the desired confidence can be defined as a confidence interval over the mean (e.g. ±5%), with smaller intervals requiring either higher concordance or a larger sample size.

In some embodiments, for discrete solution spaces (e.g. N-class classification), the consensus answer is discretized to one of the N classes (i.e. the plurality, the dimension with the largest magnitude). In such cases, confidence can be expressed as a false positive rate, somewhat analogous to a p-value, and measured according to the percentage of simulations in which the consensus answer was discretized to a different class.

FIG. 4 is a block diagram of the data classification model 116 in accordance with one or more embodiments of the present disclosure.

After a ground truth dataset 501 is established to an intermediate or final level (e.g., using the user-generated labels 103 and the weighting by the expertise analysis engine 114 to generate the likely correct input 108 as the ground truth dataset 501), the ground truth dataset 501 can be utilized inside the ground-truth system 110, or exported, to train a data classification model 116 or any number of additional applications where ground truth is useful, such as, e.g., for educational or user testing purposes, for training material, or to further evaluate users inside our system. The idea of using this existing ground truth to stratify users inside the system for skill level may appear to be self-referential but studies have indicated it is effective.

FIG. 5 depicts a block diagram of an exemplary computer-based system and platform 400 in accordance with one or more embodiments of the present disclosure. However, not all of these components may be required to practice one or more embodiments, and variations in the arrangement and type of the components may be made without departing from the spirit or scope of various embodiments of the present disclosure. In some embodiments, the illustrative computing devices and the illustrative computing components of the exemplary computer-based system and platform 400 may be configured to manage a large number of members and concurrent transactions, as detailed herein. In some embodiments, the exemplary computer-based system and platform 400 may be based on a scalable computer and network architecture that incorporates varies strategies for assessing the data, caching, searching, and/or database connection pooling. An example of the scalable architecture is an architecture that is capable of operating multiple servers.

In some embodiments, referring to FIG. 5, member computing devices 402-404 (e.g., clients) of the exemplary computer-based system and platform 400 may include virtually any computing device capable of receiving and sending a message over a network (e.g., cloud network), such as network 405, to and from another computing device, such as servers 406 and 407, each other, and the like. In some embodiments, the member devices 402-404 may be personal computers, multiprocessor systems, microprocessor-based or programmable consumer electronics, network PCs, and the like. In some embodiments, one or more member devices within member devices 402-404 may include computing devices that typically connect using a wireless communications medium such as cell phones, smart phones, pagers, walkie talkies, radio frequency (RF) devices, infrared (IR) devices, CBs, integrated devices combining one or more of the preceding devices, or virtually any mobile computing device, and the like. In some embodiments, one or more member devices within member devices 402-404 may be devices that are capable of connecting using a wired or wireless communication medium such as a PDA, POCKET PC, wearable computer, a laptop, tablet, desktop computer, a netbook, a video game device, a pager, a smart phone, an ultra-mobile personal computer (UMPC), and/or any other device that is equipped to communicate over a wired and/or wireless communication medium (e.g., NFC, RFID, NBIOT, 3G, 4G, 5G, GSM, GPRS, WiFi, WiMax, CDMA, satellite, ZigBee, etc.). In some embodiments, one or more member devices within member devices 402-404 may run one or more applications, such as Internet browsers, mobile applications, voice calls, video games, videoconferencing, and email, among others. In some embodiments, one or more member devices within member devices 402-404 may be configured to receive and to send web pages, and the like. In some embodiments, an exemplary specifically programmed browser application of the present disclosure may be configured to receive and display graphics, text, multimedia, and the like, employing virtually any web based language, including, but not limited to Standard Generalized Markup Language (SMGL), such as HyperText Markup Language (HTML), a wireless application protocol (WAP), a Handheld Device Markup Language (HDML), such as Wireless Markup Language (WML), WMLScript, XML, JavaScript, and the like. In some embodiments, a member device within member devices 402-404 may be specifically programmed by either Java, .Net, QT, C, C++ and/or other suitable programming language. In some embodiments, one or more member devices within member devices 402-404 may be specifically programmed to include or execute an application to perform a variety of possible tasks, such as, without limitation, messaging functionality, browsing, searching, playing, streaming or displaying various forms of content, including locally stored or uploaded messages, images and/or video, and/or games.

In some embodiments, the exemplary network 405 may provide network access, data transport and/or other services to any computing device coupled to it. In some embodiments, the exemplary network 405 may include and implement at least one specialized network architecture that may be based at least in part on one or more standards set by, for example, without limitation, Global System for Mobile communication (GSM) Association, the Internet Engineering Task Force (IETF), and the Worldwide Interoperability for Microwave Access (WiMAX) forum. In some embodiments, the exemplary network 405 may implement one or more of a GSM architecture, a General Packet Radio Service (GPRS) architecture, a Universal Mobile Telecommunications System (UMTS) architecture, and an evolution of UMTS referred to as Long Term Evolution (LTE). In some embodiments, the exemplary network 405 may include and implement, as an alternative or in conjunction with one or more of the above, a WiMAX architecture defined by the WiMAX forum. In some embodiments and, optionally, in combination of any embodiment described above or below, the exemplary network 405 may also include, for instance, at least one of a local area network (LAN), a wide area network (WAN), the Internet, a virtual LAN (VLAN), an enterprise LAN, a layer 3 virtual private network (VPN), an enterprise IP network, or any combination thereof. In some embodiments and, optionally, in combination of any embodiment described above or below, at least one computer network communication over the exemplary network 405 may be transmitted based at least in part on one of more communication modes such as but not limited to: NFC, RFID, Narrow Band Internet of Things (NBIOT), ZigBee, 3G, 4G, 5G, GSM, GPRS, WiFi, WiMax, CDMA, satellite and any combination thereof. In some embodiments, the exemplary network 405 may also include mass storage, such as network attached storage (NAS), a storage area network (SAN), a content delivery network (CDN) or other forms of computer or machine readable media.

In some embodiments, the exemplary server 406 or the exemplary server 407 may be a web server (or a series of servers) running a network operating system, examples of which may include but are not limited to Microsoft Windows Server, Novell NetWare, or Linux. In some embodiments, the exemplary server 406 or the exemplary server 407 may be used for and/or provide cloud and/or network computing. Although not shown in FIG. 5, in some embodiments, the exemplary server 406 or the exemplary server 407 may have connections to external systems like email, SMS messaging, text messaging, ad content providers, etc. Any of the features of the exemplary server 406 may be also implemented in the exemplary server 407 and vice versa.

In some embodiments, one or more of the exemplary servers 406 and 407 may be specifically programmed to perform, in non-limiting example, as authentication servers, search servers, email servers, social networking services servers, SMS servers, M servers, MMS servers, exchange servers, photo-sharing services servers, advertisement providing servers, financial/banking-related services servers, travel services servers, or any similarly suitable service-base servers for users of the member computing devices 402-404.

In some embodiments and, optionally, in combination of any embodiment described above or below, for example, one or more exemplary computing member devices 402-404, the exemplary server 406, and/or the exemplary server 407 may include a specifically programmed software module that may be configured to send, process, and receive information using a scripting language, a remote procedure call, an email, a tweet, Short Message Service (SMS), Multimedia Message Service (MMS), instant messaging (IM), internet relay chat (IRC), mIRC, Jabber, an application programming interface, Simple Object Access Protocol (SOAP) methods, Common Object Request Broker Architecture (CORBA), HTTP (Hypertext Transfer Protocol), REST (Representational State Transfer), or any combination thereof.

FIG. 6 depicts a block diagram of another exemplary computer-based system and platform 500 in accordance with one or more embodiments of the present disclosure. However, not all of these components may be required to practice one or more embodiments, and variations in the arrangement and type of the components may be made without departing from the spirit or scope of various embodiments of the present disclosure. In some embodiments, the member computing devices 502a, 502b through 502n shown each at least includes a computer-readable medium, such as a random-access memory (RAM) 508 coupled to a processor 510 or FLASH memory. In some embodiments, the processor 510 may execute computer-executable program instructions stored in memory 508. In some embodiments, the processor 510 may include a microprocessor, an ASIC, and/or a state machine. In some embodiments, the processor 510 may include, or may be in communication with, media, for example computer-readable media, which stores instructions that, when executed by the processor 510, may cause the processor 510 to perform one or more steps described herein. In some embodiments, examples of computer-readable media may include, but are not limited to, an electronic, optical, magnetic, or other storage or transmission device capable of providing a processor, such as the processor 510 of member computing device 502a, with computer-readable instructions. In some embodiments, other examples of suitable media may include, but are not limited to, a floppy disk, CD-ROM, DVD, magnetic disk, memory chip, ROM, RAM, an ASIC, a configured processor, all optical media, all magnetic tape or other magnetic media, or any other medium from which a computer processor can read instructions. Also, various other forms of computer-readable media may transmit or carry instructions to a computer, including a router, private or public network, or other transmission device or channel, both wired and wireless. In some embodiments, the instructions may comprise code from any computer-programming language, including, for example, C, C++, Visual Basic, Java, Python, Perl, JavaScript, etc.

In some embodiments, member computing devices 502a through 502n may also comprise a number of external or internal devices such as a mouse, a CD-ROM, DVD, a physical or virtual keyboard, a display, or other input or output devices. In some embodiments, examples of member computing devices 502a through 502n (e.g., clients) may be any type of processor-based platforms that are connected to a network 506 such as, without limitation, personal computers, digital assistants, personal digital assistants, smart phones, pagers, digital tablets, laptop computers, Internet appliances, and other processor-based devices. In some embodiments, member computing devices 502a through 502n may be specifically programmed with one or more application programs in accordance with one or more principles/methodologies detailed herein. In some embodiments, member computing devices 502a through 502n may operate on any operating system capable of supporting a browser or browser-enabled application, such as Microsoft™, Windows™, and/or Linux. In some embodiments, member computing devices 502a through 502n shown may include, for example, personal computers executing a browser application program such as Microsoft Corporation's Internet Explorer™, Apple Computer, Inc.'s Safari™, Mozilla Firefox, and/or Opera. In some embodiments, through the member computing devices 502a through 502n, users, 512a through 502n, may communicate over the exemplary network 506 with each other and/or with other systems and/or devices coupled to the network 506. As shown in FIG. 6, exemplary server devices 504 and 513 may be also coupled to the network 506. In some embodiments, one or more member computing devices 502a through 502n may be mobile clients.

In some embodiments, at least one database of exemplary databases 507 and 515 may be any type of database, including a database managed by a database management system (DBMS). In some embodiments, an exemplary DBMS-managed database may be specifically programmed as an engine that controls organization, storage, management, and/or retrieval of data in the respective database. In some embodiments, the exemplary DBMS-managed database may be specifically programmed to provide the ability to query, backup and replicate, enforce rules, provide security, compute, perform change and access logging, and/or automate optimization. In some embodiments, the exemplary DBMS-managed database may be chosen from Oracle database, IBM DB2, Adaptive Server Enterprise, FileMaker, Microsoft Access, Microsoft SQL Server, MySQL, PostgreSQL, and a NoSQL implementation. In some embodiments, the exemplary DBMS-managed database may be specifically programmed to define each respective schema of each database in the exemplary DBMS, according to a particular database model of the present disclosure which may include a hierarchical model, network model, relational model, object model, or some other suitable organization that may result in one or more applicable data structures that may include fields, records, files, and/or objects. In some embodiments, the exemplary DBMS-managed database may be specifically programmed to include metadata about the data that is stored.

In some embodiments, the illustrative computer-based systems or platforms of the present disclosure may be specifically configured to operate in a cloud computing/architecture such as, but not limiting to: infrastructure a service (IaaS), platform as a service (PaaS), and/or software as a service (SaaS). FIG. 7 and FIG. 8 illustrate schematics of exemplary implementations of the cloud computing/architecture(s) in which the illustrative computer-based systems or platforms of the present disclosure may be specifically configured to operate.

It is understood that at least one aspect/functionality of various embodiments described herein can be performed in real-time and/or dynamically. As used herein, the term “real-time” is directed to an event/action that can occur instantaneously or almost instantaneously in time when another event/action has occurred. For example, the “real-time processing,” “real-time computation,” and “real-time execution” all pertain to the performance of a computation during the actual time that the related physical process (e.g., a user interacting with an application on a mobile device) occurs, in order that results of the computation can be used in guiding the physical process.

As used herein, the term “dynamically” and term “automatically,” and their logical and/or linguistic relatives and/or derivatives, mean that certain events and/or actions can be triggered and/or occur without any human intervention. In some embodiments, events and/or actions in accordance with the present disclosure can be in real-time and/or based on a predetermined periodicity of at least one of: nanosecond, several nanoseconds, millisecond, several milliseconds, second, several seconds, minute, several minutes, hourly, several hours, daily, several days, weekly, monthly, etc.

In some embodiments, exemplary inventive, specially programmed computing systems and platforms with associated devices are configured to operate in the distributed network environment, communicating with one another over one or more suitable data communication networks (e.g., the Internet, satellite, etc.) and utilizing one or more suitable data communication protocols/modes such as, without limitation, IPX/SPX, X.25, AX.25, AppleTalk™, TCP/IP (e.g., HTTP), near-field wireless communication (NFC), RFID, Narrow Band Internet of Things (NBIOT), 3G, 4G, 5G, GSM, GPRS, WiFi, WiMax, CDMA, satellite, ZigBee, and other suitable communication modes. In some embodiments, the NFC can represent a short-range wireless communications technology in which NFC-enabled devices are “swiped,” “bumped,” “tap” or otherwise moved in close proximity to communicate. In some embodiments, the NFC could include a set of short-range wireless technologies, typically requiring a distance of 10 cm or less. In some embodiments, the NFC may operate at 13.56 MHz on ISO/IEC 18000-3 air interface and at rates ranging from 106 kbit/s to 424 kbit/s. In some embodiments, the NFC can involve an initiator and a target; the initiator actively generates an RF field that can power a passive target. In some embodiment, this can enable NFC targets to take very simple form factors such as tags, stickers, key fobs, or cards that do not require batteries. In some embodiments, the NFL's peer-to-peer communication can be conducted when a plurality of NFC-enabled devices (e.g., smartphones) within close proximity of each other.

The material disclosed herein may be implemented in software or firmware or a combination of them or as instructions stored on a machine-readable medium, which may be read and executed by one or more processors. A machine-readable medium may include any medium and/or mechanism for storing or transmitting information in a form readable by a machine (e.g., a computing device). For example, a machine-readable medium may include read only memory (ROM); random access memory (RAM); magnetic disk storage media; optical storage media; flash memory devices; electrical, optical, acoustical or other forms of propagated signals (e.g., carrier waves, infrared signals, digital signals, etc.), and others.

As used herein, the terms “computer engine” and “engine” identify at least one software component and/or a combination of at least one software component and at least one hardware component which are designed/programmed/configured to manage/control other software and/or hardware components (such as the libraries, software development kits (SDKs), objects, etc.).

Examples of hardware elements may include processors, microprocessors, circuits, circuit elements (e.g., transistors, resistors, capacitors, inductors, and so forth), integrated circuits, application specific integrated circuits (ASIC), programmable logic devices (PLD), digital signal processors (DSP), field programmable gate array (FPGA), logic gates, registers, semiconductor device, chips, microchips, chip sets, and so forth. In some embodiments, the one or more processors may be implemented as a Complex Instruction Set Computer (CISC) or Reduced Instruction Set Computer (RISC) processors; x86 instruction set compatible processors, multi-core, or any other microprocessor or central processing unit (CPU). In various implementations, the one or more processors may be dual-core processor(s), dual-core mobile processor(s), and so forth.

Computer-related systems, computer systems, and systems, as used herein, include any combination of hardware and software. Examples of software may include software components, programs, applications, operating system software, middleware, firmware, software modules, routines, subroutines, functions, methods, procedures, software interfaces, application program interfaces (API), instruction sets, computer code, computer code segments, words, values, symbols, or any combination thereof. Determining whether an embodiment is implemented using hardware elements and/or software elements may vary in accordance with any number of factors, such as desired computational rate, power levels, heat tolerances, processing cycle budget, input data rates, output data rates, memory resources, data bus speeds and other design or performance constraints.

One or more aspects of at least one embodiment may be implemented by representative instructions stored on a machine-readable medium which represents various logic within the processor, which when read by a machine causes the machine to fabricate logic to perform the techniques described herein. Such representations, known as “IP cores” may be stored on a tangible, machine readable medium and supplied to various customers or manufacturing facilities to load into the fabrication machines that make the logic or processor. Of note, various embodiments described herein may, of course, be implemented using any appropriate hardware and/or computing software languages (e.g., C++, Objective-C, Swift, Java, JavaScript, Python, Perl, QT, etc.).

In some embodiments, one or more of illustrative computer-based systems or platforms of the present disclosure may include or be incorporated, partially or entirely into at least one personal computer (PC), laptop computer, ultra-laptop computer, tablet, touch pad, portable computer, handheld computer, palmtop computer, personal digital assistant (PDA), cellular telephone, combination cellular telephone/PDA, television, smart device (e.g., smart phone, smart tablet or smart television), mobile internet device (MID), messaging device, data communication device, and so forth.

As used herein, the term “server” should be understood to refer to a service point which provides processing, database, and communication facilities. By way of example, and not limitation, the term “server” can refer to a single, physical processor with associated communications and data storage and database facilities, or it can refer to a networked or clustered complex of processors and associated network and storage devices, as well as operating software and one or more database systems and application software that support the services provided by the server. Cloud servers are examples.

In some embodiments, as detailed herein, one or more of the computer-based systems of the present disclosure may obtain, manipulate, transfer, store, transform, generate, and/or output any digital object and/or data unit (e.g., from inside and/or outside of a particular application) that can be in any suitable form such as, without limitation, a file, a contact, a task, an email, a message, a map, an entire application (e.g., a calculator), data points, and other suitable data. In some embodiments, as detailed herein, one or more of the computer-based systems of the present disclosure may be implemented across one or more of various computer platforms such as, but not limited to: (1) Linux, (2) Microsoft Windows, (3) OS X (Mac OS), (4) Solaris, (5) UNIX (6) VMWare, (7) Android, (8) Java Platforms, (9) Open Web Platform, (10) Kubernetes or other suitable computer platforms. In some embodiments, illustrative computer-based systems or platforms of the present disclosure may be configured to utilize hardwired circuitry that may be used in place of or in combination with software instructions to implement features consistent with principles of the disclosure. Thus, implementations consistent with principles of the disclosure are not limited to any specific combination of hardware circuitry and software. For example, various embodiments may be embodied in many different ways as a software component such as, without limitation, a stand-alone software package, a combination of software packages, or it may be a software package incorporated as a “tool” in a larger software product.

For example, exemplary software specifically programmed in accordance with one or more principles of the present disclosure may be downloadable from a network, for example, a website, as a stand-alone product or as an add-in package for installation in an existing software application. For example, exemplary software specifically programmed in accordance with one or more principles of the present disclosure may also be available as a client-server software application, or as a web-enabled software application. For example, exemplary software specifically programmed in accordance with one or more principles of the present disclosure may also be embodied as a software package installed on a hardware device.

In some embodiments, illustrative computer-based systems or platforms of the present disclosure may be configured to handle numerous concurrent users that may be, but is not limited to, at least 100 (e.g., but not limited to, 100-999), at least 1,000 (e.g., but not limited to, 1,000-9,999), at least 10,000 (e.g., but not limited to, 10,000-99,999), at least 100,000 (e.g., but not limited to, 100,000-999,999), at least 1,000,000 (e.g., but not limited to, 1,000,000-9,999,999), at least 10,000,000 (e.g., but not limited to, 10,000,000-99,999,999), at least 100,000,000 (e.g., but not limited to, 100,000,000-999,999,999), at least 1,000,000,000 (e.g., but not limited to, 1,000,000,000-999,999,999,999), and so on.

In some embodiments, illustrative computer-based systems or platforms of the present disclosure may be configured to output to distinct, specifically programmed graphical user interface implementations of the present disclosure (e.g., a desktop, a web app., etc.). In various implementations of the present disclosure, a final output may be displayed on a displaying screen which may be, without limitation, a screen of a computer, a screen of a mobile device, or the like. In various implementations, the display may be a holographic display. In various implementations, the display may be a transparent surface that may receive a visual projection. Such projections may convey various forms of information, images, or objects. For example, such projections may be a visual overlay for a mobile augmented reality (MAR) application.

As used herein, the term “user mobile device” “mobile computing device” or “mobile electronic device,” or the like, may refer to any portable electronic device that may or may not be enabled with location tracking functionality (e.g., MAC address, Internet Protocol (IP) address, or the like). For example, a mobile electronic device can include, but is not limited to, a mobile phone, Personal Digital Assistant (PDA), Blackberry™, Pager, Smartphone, or any other reasonable mobile electronic device.

As used herein, terms “cloud,” “Internet cloud,” “cloud computing,” “cloud architecture,” and similar terms correspond to at least one of the following: (1) a large number of computers connected through a real-time communication network (e.g., Internet); (2) providing the ability to run a program or application on many connected computers (e.g., physical machines, virtual machines (VMs)) at the same time; (3) network-based services, which appear to be provided by real server hardware, and are in fact served up by virtual hardware (e.g., virtual servers), simulated by software running on one or more real machines (e.g., allowing to be moved around and scaled up (or down) on the fly without affecting the end user).

In some embodiments, the illustrative computer-based systems or platforms of the present disclosure may be configured to securely store and/or transmit data by utilizing one or more of encryption techniques (e.g., private/public key pair, Triple Data Encryption Standard (3DES), block cipher algorithms (e.g., IDEA, RC2, RC5, CAST and Skipjack), cryptographic hash algorithms (e.g., MD5, RIPEMD-160, RTR0, SHA-1, SHA-2, Tiger (TTH), WHIRLPOOL, RNGs).

The aforementioned examples are, of course, illustrative and not restrictive.

As used herein, the term “user” shall have a meaning of at least one user. In some embodiments, the terms “user”, “subscriber” “consumer” or “customer” should be understood to refer to a user of an application or applications as described herein and/or a consumer of data supplied by a data provider. By way of example, and not limitation, the terms “user” or “subscriber” can refer to a person who receives data provided by the data or service provider over the Internet in a browser session or can refer to an automated software application which receives the data and stores or processes the data.

Throughout the specification, the following terms take the meanings explicitly associated herein, unless the context clearly dictates otherwise. The phrases “in one embodiment” and “in some embodiments” as used herein do not necessarily refer to the same embodiment(s), though it may. Furthermore, the phrases “in another embodiment” and “in some other embodiments” as used herein do not necessarily refer to a different embodiment, although it may. Thus, as described below, various embodiments may be readily combined, without departing from the scope or spirit of the present disclosure.

In addition, the term “based on” is not exclusive and allows for being based on additional factors not described, unless the context clearly dictates otherwise. In addition, throughout the specification, the meaning of “a,” “an,” and “the” include plural references. The meaning of “in” includes “in” and “on.”

As used herein, the terms “and” and “or” may be used interchangeably to refer to a set of items in both the conjunctive and disjunctive in order to encompass the full description of combinations and alternatives of the items. By way of example, a set of items may be listed with the disjunctive “or”, or with the conjunction “and.” In either case, the set is to be interpreted as meaning each of the items singularly as alternatives, as well as any combination of the listed items.

While one or more embodiments of the present disclosure have been described, it is understood that these embodiments are illustrative only, and not restrictive, and that many modifications may become apparent to those of ordinary skill in the art, including that various embodiments of the inventive methodologies, the illustrative systems and platforms, and the illustrative devices described herein can be utilized in any combination with each other. Further still, the various steps may be carried out in any desired order (and any desired steps may be added and/or any desired steps may be eliminated).

What is claimed is:

1. A method comprising:
receiving, by at least one processor, user-generated labels for each sample of at least one sample;
wherein the user-generated labels are associated with at least one user profile of at least one user;
receiving, by the at least one processor, the at least one user profile;
producing, by the at least one processor, a most likely label for each sample based at least in part on a weighting of each user-generated label of the user-generated labels according to user attributes represented in each user profile of the at least one user profile associated with each user-generated label;
recording, by the at least one processor, each most likely label for each sample in a ground-truth dataset associated with each sample to identify a ground truth of the at least one sample; and
providing, by the at least one processor, each most likely label and each sample to a machine learning model to train the machine learning model according to a difference between a predicted label for each sample and each most likely label.

2. The method of claim 1, further comprising:
determining, by the at least one processor, a distance from a consensus of each user-generated label of the user-generated labels based at least in part on a distance measure of each user-generated label from the most likely label; and
determining, by the at least one processor, a user skill level for each user profile based on the distance from the consensus of each user-generated label associated with each user profile.

3. The method of claim 1, further comprising:
accessing, by the at least one processor, prior distances from consensus for each user profile, wherein the prior distances from the consensus comprises a distance measure of each prior user-generated label from each prior most likely label;
determining, by the at least one processor, an aggregated distance for each user profile based on a statistical aggregation of the prior distances of each user profile; and
generating, by the at least one processor, the weighting of each user-generated label of the user-generated labels according to user attributes represented in each user profile of the at least one user profile associated with each user-generated label.

4. The method of claim 3, wherein the statistical aggregation comprises an average.

5. The method of claim 3, further comprising:
determining, by the at least one processor, a weighted mean for each sample according to a weighting of the user-generated labels of each sample;
determining, by the at least one processor, a sampling distribution of the user-generated labels and the weighted mean for each sample; and
determining, by the at least one processor, a confidence of each most likely label for each sample based on a standard deviation of the sampling distribution.

6. The method of claim 5, further comprising filtering, by the at least one processor, each likely label based on a comparison of the confidence of each most likely label to a threshold confidence.

7. The method of claim 5, wherein the confidence of each most likely label comprises a confidence interval.

8. The method of claim 5, wherein the confidence of each most likely label comprises a false positive rate.

9. The method of claim 1, wherein each sample comprises biological sample data.

10. The method of claim 9, wherein the biological sample data comprises medical imagery.

11. A system comprising:
at least one processor in communication with at least one memory configured to access instructions in the at least one memory that cause the at least one processor to perform steps to:
receive user-generated labels for each medical sample of at least one sample;
wherein the user-generated labels are associated with at least one user profile of at least one user;
receive the at least one user profile;
produce a most likely label for each medical sample based at least in part on a weighting of each user-generated label of the user-generated labels according to user attributes represented in each user profile of the at least one user profile associated with each user-generated label; and
record each most likely label for each medical sample in a ground-truth dataset associated with each medical sample to identify a ground truth of the at least one sample.

12. The system of claim 11, wherein the instructions cause the at least one processor to further perform steps to:
determine a distance from a consensus of each user-generated label of the user-generated labels based at least in part on a distance measure of each user-generated label from the most likely label; and
determine a user skill level for each user profile based on the distance from the consensus of each user-generated label associated with each user profile.

13. The system of claim 11, wherein the instructions cause the at least one processor to further perform steps to:
access prior distances from consensus for each user profile, wherein the prior distances from the consensus comprises a distance measure of each prior user-generated label from each prior most likely label;
determine an aggregated distance for each user profile based on a statistical aggregation of the prior distances of each user profile; and
generate the weighting of each user-generated label of the user-generated labels according to user attributes represented in each user profile of the at least one user profile associated with each user-generated label.

14. The system of claim 13, wherein the statistical aggregation comprises an average.

15. The system of claim 13, wherein the instructions cause the at least one processor to further perform steps to:
determine a weighted mean for each sample according to a weighting of the user-generated labels of each sample;
determine a sampling distribution of the user-generated labels and the weighted mean for each sample; and
determine a confidence of each most likely label for each sample based on a standard deviation of the sampling distribution.

16. The system of claim 15, wherein the instructions cause the at least one processor to further perform steps to filter each likely label based on a comparison of the confidence of each most likely label to a threshold confidence.

17. The system of claim 15, wherein the confidence of each most likely label comprises a confidence interval.

18. The system of claim 15, wherein the confidence of each most likely label comprises a false positive rate.

19. The system of claim 11, wherein each sample comprises biological sample data.

20. The system of claim 19, wherein the biological sample data comprises medical imagery.
